(19) United States
(12) Patent Application Publication
(10) Pub. No.: US 2018/0215865 A1
(43) Pub. Date: Aug. 2, 2018

(54) TELECHELIC PREPOLYMERS AND REACTION PRODUCTS THEREOF

(71) Applicant: Regents of the University of Minnesota, Minneapolis, MN (US)

(72) Inventors: Marc Hillmyer, Minneapolis, MN (US); Yanzhao Wang, Minneaplis, MN (US)

(73) Assignee: Regents of the University of Minnesota, Minneapolis, MN (US)

(21) Appl. No.: 15/750,533

(22) PCT Filed: Jul. 14, 2016
(86) PCT No.: PCT/US2016/042231

§ 371 Date: Feb. 6, 2018

Related U.S. Application Data
(60) Provisional application No. 62/201,704, filed on Aug. 6, 2015.

(51) Int. Cl. C08G 61/08; C07C 1/22; C07C 13/263; C08G 18/76; C08G 18/64

(52) U.S. Cl. CPC C08G 61/08; C07C 1/22; C07C 13/263; C08G 18/7671; C08G 18/64; C08G 2261/80; C08G 2261/1412; C08G 2261/1644; C08G 2261/3322; C08G 2261/418; C08G 2261/724; C07C 2601/18

(57) ABSTRACT

Gem-dialkyl cyclooctene monomers, telechelic prepolymers prepared by ring opening metathesis polymerization of the monomers, and polymers such as polyurethanes comprising the reaction product of the prepolymer and a co-monomer such as a polyisocyanate.

### CROSS-REFERENCE TO RELATED APPLICATIONS

This application claims the benefit of U.S. Provisional Application Ser. No. 62/201,704, filed Aug. 6, 2015, the disclosure of which is incorporated herein by reference in its entirety.

### TECHNICAL FIELD

This invention relates to preparing telechelic prepolymers and reaction products of these prepolymers.

### BACKGROUND

Polymers with repeating units containing geminal dialkyl groups are widely applied in a variety of industrial areas because of their attractive properties: extremely low permeability, excellent oxidative stability, and chemical resistance. Some polymers in this class of materials have also been shown to be biocompatible and approved by the Food and Drug Administration (FDA) for food-related applications. Thermoplastic polyurethane (PU) elastomers containing geminal dialkyl groups represent a particularly interesting class of biomaterials and have attracted much attention. Due in part to the inclusion of geminal dialkyl groups, the oxidative, hydrolytic, and thermal stability of these PU's, along with barrier properties, are far superior to conventional PUs containing polyesters, polycarbonates and polyethers as soft segments.

### SUMMARY

In one aspect, there is described a gem-dialkyl cyclooctene monomer where each alkyl group, independently, is an acyclic alkyl group or the two alkyl groups together form a cyclic alkyl group. The monomer may contain one or more pairs of gem-dialkyl groups. In some embodiments, each acyclic alkyl group may have between 1 and 20 carbon atoms, inclusive. For example, each acyclic alkyl group could be a methyl, ethyl, or propyl group. In some embodiments, the two gem alkyl groups together form a cyclic alkyl group having between 3 and 12 carbon atoms, inclusive. An example of a gem-dialkyl cyclooctene monomer is (Z)-5,5-dialkylcyclooct-1-ene monomer where each alkyl group may, e.g., be a methyl group.

In a second aspect, there is described a telechelic prepolymer comprising the ring opening metathesis polymerization (“ROMP”) product of the above-described gem-dialkyl cyclooctene monomer. Examples of suitable ring opening metathesis polymerization catalysts include Grubbs second generation catalysts. One or more cyclic olefin monomers different from the gem-dialkyl cyclooctene monomer may also be included in the polymerization reaction. The cyclic olefin monomer may have between 3 and 12 carbon atoms, inclusive. A useful example is cis-cyclooctene. The polymerization process may include (a) polymerizing the gem-dialkyl cyclooctene monomer in the presence of a ring opening metathesis polymerization catalyst and a symmetric acyclic olefin chain transfer agent having a pair of functional end groups to form an unsaturated precursor having a pair of functional end groups; and (b) hydrogenating the precursor to form the telechelic prepolymer. In some embodiments, the functional groups of the precursor may be further reacted, e.g., hydrolyzed, to form different functional groups. Examples of suitable chain transfer agents include agents selected from the group consisting of:

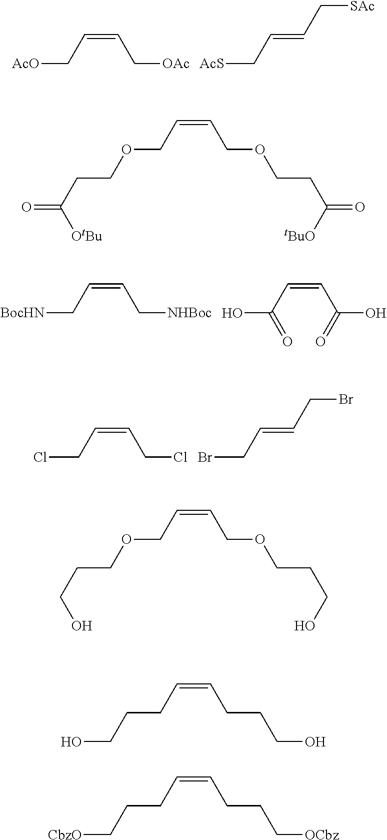

In one embodiment, the chain transfer agent is an unsaturated diacetoxy-functional compounds such as 1,4-diacetoxy-cis-2-butene.

In another embodiment, the chain transfer agent has the formula

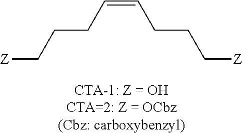

where Z=OH or OCbz.

The prepolymers can be used to prepare other polymers. For example, hydroxy-functional prepolymers such as hydroxy-telechelic hydrogenated poly(5,5-dimethylcyclooct-1-ene) prepolymers can be reacted with polyisocyanates to form polyurethanes.

The details of one or more embodiments of the invention are set forth in the description below. Other features, objects, and advantages of the invention will be apparent from the description and from the claims.

### DETAILED DESCRIPTION

Gem-dialkyl cyclooctene monomers and their use in preparing telechelic prepolymers via ring opening metathesis polymerization (“ROMP”) using a ROMP catalyst, are described in the Summary of the Invention above. The following examples describe the preparation of (Z)-5,5-dialkylcyclooct-1-ene monomer and hydroxy-telechelic poly(5,5-dimethylcyclooct-1-ene) prepolymers prepared from this monomer. The prepolymers may be reacted with polyisocyanates to form segmented polyurethanes in which the prepolymers form the soft block of the polyurethane.

### Examples

Materials. Ethyl acetate (ACS grade), hexanes (ACS grade), and diethyl ether (anhydrous) were purchased from Fisher Scientific and used without further purification. Anhydrous tetrahydrofuran and dichloromethane were obtained from solvent purification system directly. All commercially available reactants/reagents were purchased from Aldrich and used without further purification. Reactions were monitored by thin layer chromatography (TLC) using silicycle pre-coated silica gel plates. Flash column chromatography was performed over silicycle silica gel (230-400 mesh).

Instruments.

¹H NMR and ¹³C NMR spectra were recorded on a Varian INOVA-500 spectrometer, Bruker AV500 spectrometer and a Bruker HD500 spectrometer using residue solvent peaks as internal standards; CDCl3 was used as the solvent. High-resolution mass spectral data (HRMS) was collected on an Agilent Technologies 7200 Accuate-Mass Q-TOF GC/MS using EI conditions. M_{n,NMR} was determined by ¹H NMR end group analysis. M_{n,SEC} was determined on a Hewlett-Packard 1100 series liquid chromatograph fitted with a Hewlett-Packard 1047A refractive index detector and three PLgel columns (Polymer Laboratories columns with 500, 103, and 104 Å pore sizes), which were calibrated with polystyrene standards, with chloroform as the eluent at a flow rate of 1 mL/min at 35° C. M_{n,LS-SEC} was determined on a system includes a Wyatt OPTILAB RI detector, a Wyatt multiangle light scattering detector (MALS), and three Phenogel columns (Phenomenex of 103, 104, and 105 Å pore sizes). The columns were at ambient temperature, and the RI detector was set at 40° C.; THF was used as the eluent at a flow rate of 1 mL/min. Differential Scanning calorimetry (DSC) measurements were performed using a TA Instruments Q1000 with N₂ as the purge gas at the rate of 10° C./min. Thermal transition temperatures were determined from the second heating after annealing above the glass transition or melting temperatures for at least 1 min to erase thermal history.

### Example 1—Synthesis of Monomer 5

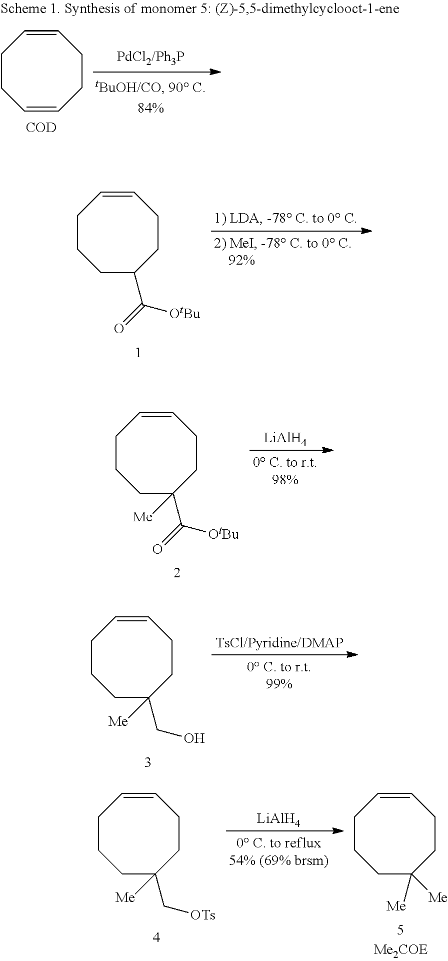

### (a) Synthesis of tert-Butyl (Z)-cyclooct-4-ene-1-carboxylate 1

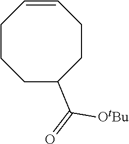 [SMILES: CC(C)(C)OC(=O)C1CC/C=C\CCC1]

This known compound was prepared via a slightly different procedure from Wagener and coworkers (Lehman, S. E.; Wagener, K. B.; Baugh, L. S.; Rucker, S. P.; Schulz, D. N.; Varma-Nair, M.; Berluche, E. Macromolecules 2007, 40, 2643) and the spectral data were in accordance with literature data. In a 1 L high pressure reactor (Series 4520 Bench Top Reactors, 1 L, Parr Instrument Company) were placed palladium (II) chloride (2 g, 11.3 mmol), triphenylphosphine (12 g, 45.7 mmol), tert-butyl alcohol (69 mL, 718 mmol), 1,5-cyclooctadiene (COD, 140 mL, 1141 mmol), and toluene (69 mL). The reactor was sealed and then pressurized to 600 psi with carbon monoxide and then vented down to 25-30 psi. This procedure was repeated two more times and then the reactor was pressurized to 400 psig and heated to 90° C. with fast stirring. After the system had equilibrated at this temperature, the reactor was charged with additional carbon monoxide to a pressure of 660 psi. After 24 hours, the pressure dropped significantly and the reactor was then re-pressurized to 660 psig and stirred for another 24 hours. Then the reactor was cooled to room temperature, vented, and disassembled. The yellow solution was filtered with Celite and washed with toluene, and the volatiles were removed in vacuo. The crude product was purified by fractional vacuum distillation yielding a clear colorless oil (126 g, 84% yield, b.p.=67-70° C. at 200-250 mTorr).

¹H NMR (500 MHz, CDCl₃) δ 5.73-5.57 (m, 2H), 2.42-2.28 (m, 2H), 2.20-2.02 (m, 3H), 1.97 (dt, J₁=14.7 Hz, J₂=4.0 Hz, 1H), 1.86-1.79 (m, 1H), 1.74-1.67 (m, 1H), 1.62-1.49 (m, 2H), 1.42 (s, 10H).

### (b) Synthesis of tert-Butyl (Z)-1-methylcyclooct-4-ene-1-carboxylate 2

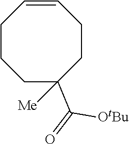 [SMILES: CC(C)(C)OC(=O)C1(C)CC/C=C\CCC1]

This known compound was prepared via a slightly different procedure from Coates and coworkers (Robertson, N. J.; Kostalik, H. A.; Clark, T. J.; Mutolo, P. F.; Abram, H. D.; Coates, G. W. J Am. Chem. Soc. 2010, 132, 3400). A freshly prepared LDA solution (476 mmol diisopropylamine, 400 ml 2.5 M n-butyllithium in hexanes and 800 mL anhydrous tetrahydrofuran) was cooled to −78° C. A solution of compound 1 (66.7 g, 317 mmol) in 150 mL dry tetrahydrofuran was slowly added to the LDA solution over 30 minutes via cannula. The reaction was stirred at −78° C. for 15 minutes and then slowly warmed up to 0° C. over 30 minutes by removing the acetone-dry ice bath. Methyl iodide (41.6 mL, 667 mmol) was added dropwise and the mixture was stirred for 60 minutes at 0° C. 125 mL of 4 M hydrochloric acid was slowly added at 0° C., followed by extraction with diethyl ether (3×500 mL). The extracts were combined, washed with saturated sodium bicarbonate (150 mL), saturated sodium chloride (150 mL), and then dried with magnesium sulfate. The solvents were removed in vacuo and a yellow oil was yielded. The crude product was further purified by fractional vacuum distillation affording a clear, slightly yellow oil (65 g, 92% yield, b.p.=65-68° C. at 250 mTorr).

¹H NMR (500 MHz, CDCl₃) δ 5.73-5.63 (m, 1H), 5.52-5.40 (m, 1H), 2.37-2.20 (m, 3H), 2.20-2.00 (m, 2H), 1.81-1.65 (m, 2H), 1.65-1.34 (m, 13H), 1.14 (s, 3H).

¹H NMR (500 MHz, C₆D₆) δ 5.72-5.65 (m, 1H), 5.53-5.45 (m, 1H), 2.45-2.35 (m, 2H), 2.35-2.27 (m, 1H), 2.10-1.97 (m, 2H), 1.88-1.82 (m, 1H), 1.82-1.74 (m, 1H), 1.68-1.61 (m, 1H), 1.52 (m, 1H), 1.42-1.37 (m, 1H), 1.35 (s, 9H), 1.13 (s, 3H).

¹³C NMR (125 MHz, C₆D₆) δ 177.12, 132.84, 127.44, 79.73, 47.13, 36.86, 33.64, 28.63, 28.58, 26.69, 25.97, 25.52.

### (c) Synthesis of (Z)-(1-Methylcyclooct-4-en-1-yl)methanol 3

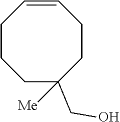 [SMILES: CC1(CO)CC/C=C\CCC1]

Lithium aluminum hydride (20.9 g, 550 mmol) was placed in a flame-dried two-neck flask under nitrogen and cooled to 0° C. Dry tetrahydrofuran (800 mL) was transferred to the flask via cannula and then a solution of compound 2 (61.8 g, 275 mmol) in 100 mL dry tetrahydrofuran was slowly added with an addition funnel. This reaction was stirred at 0° C. for 1 hour and then slowly warmed up to room temperature. After 6 hours, the solution was cooled back to 0° C. and diluted with 1 L diethyl ether; 21 mL water was added extremely slowly to quench the reaction and then followed by 21 mL 15% sodium hydroxide aqueous solution and 63 mL water. The grey mixture was warmed up to room temperature and stirred for 15 minutes, forming a white slurry. Magnesium sulfate was added and the mixture was filtered through Celite and rinsed with diethyl ether to afford clear solution, which was concentrated in vacuo to yield the alcohol 3 as a slightly yellow oil (41.6 g, 98%).

¹H NMR (500 MHz, CDCl₃) δ 5.73-5.63 (m, 1H), 5.50-5.40 (m, 1H), 3.32 (s, 2H), 2.35-2.10 (m, 4H), 1.67-1.30 (m, 7H), 0.94 (s, 3H).

### (d) Synthesis of (Z)-(1-Methylcyclooct-4-en-1-yl)methyl 4-methylbenzenesulfonate 4

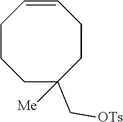 [SMILES: Cc1ccc(S(=O)(=O)OCC2(C)CC/C=C\CCC2)cc1]

An oven-dried round-bottom flask was charged with alcohol 3 (20.8 g, 135 mmol), 4-dimethylaminopyridine (1 g, 8.2 mmol) and dry pyridine (100 mL), and then cooled to 0° C. A solution of 4-toluenesulfonyl chloride (38.6 g, 202 mmol) in dry dichloromethane was added dropwise to the reaction via an addition funnel. The ice bath was then removed and the reaction was stirred for 12 h. Saturated sodium bicarbonate aqueous solution (200 mL) was added slowly at 0° C., and the mixture was stirred for 1 hour to quench the excess 4-toluenesulfonyl chloride, followed by extraction with diethyl ether (3×250 mL). The extracts were combined, washed with 4 M hydrochloric acid (250 mL), saturated sodium chloride (150 mL), and then dried with magnesium sulfate. The crude product was afforded in quantitative yield and used directly in the next step after filtration through a silica plug and subsequent removal of solvents in vacuo.

¹H NMR (500 MHz, CDCl₃) δ 7.78 (d, J=8.3 Hz, 1H), 7.34 (d, J=8.2 Hz, 2H), 5.70-5.55 (m, 1H), 5.55-5.35 (m, 1H), 3.39 (s, 2H), 2.45 (s, 3H), 2.27-1.95 (4H, m), 1.66-1.39 (m, 5H), 1.39-1.25 (m, 1H), 0.91 (s, 3H).

### (e) Synthesis of (Z)-5,5-Dimethylcyclooct-1-ene 5 (Me₂COE)

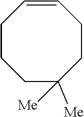

Lithium aluminum hydride (20.5 g, 540 mmol) was placed in a flame-dried two-neck flask under nitrogen and cooled to 0° C. Dry tetrahydrofuran (400 mL) was transferred to the flask via cannula and then a solution of compound 2 (27.7 g, 90 mmol) in 100 mL dry tetrahydrofuran was slowly added with an addition funnel. This reaction was stirred at 0° C. for 30 minutes and heated to gently reflux. After 12 hours, the solution was cooled back to 0° C. and diluted with 500 L diethyl ether; 20.5 mL water was added extremely slowly to quench the reaction and then followed by 20.5 mL 15% sodium hydroxide aqueous solution and 61.5 mL water. The grey mixture was warmed up to room temperature and stirred for 15 minute forming a white slurry. Magnesium sulfate was added and the mixture was filtered through Celite and rinsed with diethyl ether to afford clear solution, which was concentrated in vacuo to give a slightly yellow residue. The residue was filter through silica gel plug with pentane and the final monomer Me₂COE 5 was achieved in 54% yield (6.7 g, 48.6 mmol) after the removal of solvent in vacuo. Alcohol 3 (3 g, 19.5 mmol) was also recovered in 22% yield after flash chromatography.

¹H NMR (500 MHz, CDCl₃) δ 5.71-5.63 (m, 1H), 5.48-5.39 (m, 1H), 2.22 (q, J=7.5 Hz, 2H), 2.18-2.13 (m, 2H), 1.61-1.49 (m, 4H), 1.40-1.31 (m, 2H), 0.92 (6H, s).

¹³C NMR (125 MHz, CDCl₃) δ 132.43, 125.81, 39.96, 35.17, 34.10, 29.99, 26.11, 24.60, 24.49.

HRMS(EI): m/z calcd for C₈H₁₀ [M⁺]: 138.1409, found: 138.1400.

IR (neat): 3005, 2951, 2925, 2866, 1483, 1446, 1363, 736, 726, 654.

### Example 2—Synthesis of Telechelic LLDPE HP(Me₂COE)-OH 7

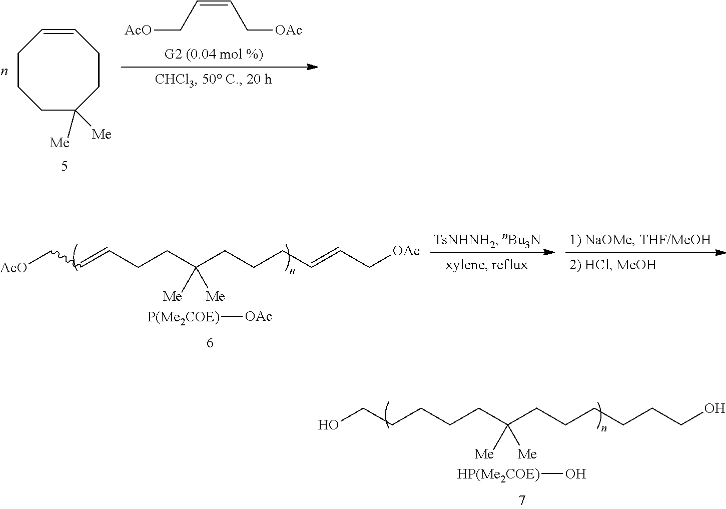

Step 1: ROMP

A 20 ml vial with a Teflon coated magnetic stir-bar was capped with a rubber septa. The vial was flame-dried under high vacuum then back-filled with argon; this evacuation fill cycle was repeated two more times. Anhydrous chloroform (3.3 mL), Me₂COE 5 (1.38 g, 10 mmol) and 1,4-diacetoxy-cis-2-butene (45.3 μL, 0.286 mmol) were added to the flask via syringe and the system was purged with argon for 5 minutes, and then immersed in an oil bath at 50° C. G2 catalyst (3.4 mg) was added via syringe as a solution in 0.3 mL of anhydrous-degassed chloroform. After 20 hours, the reaction was cooled to room temperature, quenched with 0.1 ml of ethyl vinyl ether, stirred for an additional 15 minutes and then cooled to 0° C. The polymer was precipitated by adding methanol to the solution and the methanol was decanted to leave viscous beige liquid polymer after stirring for 1 hour. The polymer was dissolved in 10 mL of dichloromethane and then 5 mg of butylated hydroxytoluene (BHT) was added. The solvent was removed in vacuo and the polymer was dried under high vacuum at 30° C. The dried polymer PMe₂COE-OAc was obtained as a viscous clear yellowish liquid with a yield of 90% (1.24 g) and was then characterized by ¹H NMR, ¹³C NMR, SEC, TGA and DSC.

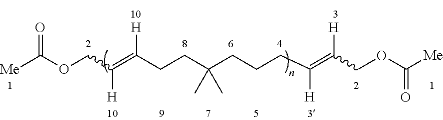

¹H NMR (500 MHz, CDCl₃) δ 5.83-5.73 (H_{3′}, m, 0.05H), 5.61-5.52 (H₃, m, 0.06H), 5.47-5.20 (H₁₀, m, 2H), 4.62 (H_{2-cis}, d, J=6.9 Hz, 0.01H), 4.51 (Hz-trans, t, J=6.1 Hz, 0.11H), 2.06 (H₁, s, 0.18H), 2.05-1.78 (H_{4,9}, m, 4H), 1.36-1.08 (H_{5,6,8}, m, 6H), 1.00-0.70 (H₇, two singles, 6H).

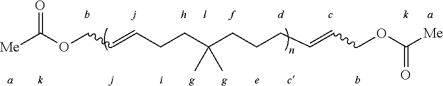

¹³C NMR (125 MHz, CDCl₃) δ 170.81 (Cₖ), 137.39/136.67 (C_{c′}), 131.06-129.44 (Cⱼ), 123.76/123.23 (C_{c}), 65.35/65.30 (C_{b}), 41.98-41.51, 33.54, 33.50, 32.74/32.62 (Cₗ), 28.08, 27.97, 27.35/27.23 (C_{g}), 24.23, 24.18, 24.14, 21.03 (Cₐ).

IR (neat): 2954, 2928, 1745, 1469, 1384, 1364, 1228, 965, 718.

Step 2: Hydrogenation and Deprotection

A mixture of PMe₂COE-OAc (1.10 g, 8 mmol of olefin), p-toluenesulfonhydrazide (5.0 g, 25 mmol), tributylamine (5.2 g, 28 mmol), small amount of BHT (ca. 5 mg), and xylene (50 mL) was refluxed for 6 hours, and then allowed to cool to room temperature. The solvent of reaction mixture was removed in vacuo and cold methanol was poured into mixture to precipitate the polymer. The polymer was isolated by decantation and purified by repeating the precipitation using chloroform/methanol system. The polymer was dried under high vacuum at 30° C. overnight to afford hydrogenated poly(5Me₂COE) as a viscous liquid. 12% of the OAc end groups were converted to OH groups under the above reaction conditions. The above polymer was then dissolved in 15 mL tetrahydrofuran and cooled to 0° C. A 500 mg sodium methoxide in methanol (25 wt. %) was added to the THF solution and this reaction was stirred for 6 hours at 0° C. The reaction mixture was acidified by slightly acidic methanol and stirred for 1 hour at room temperature. The mixture was decanted, and the polymer was washed with methanol. After the final wash the polymer HPMe₂COE-OH was dried under high vacuum at 30° C. to give a viscous clear liquid with an 80% overall yield of two steps (0.88 g) and was then characterized by ¹H NMR, ¹³C NMR, SEC, TGA and DSC.

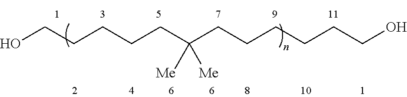

¹H NMR (500 MHz, CDCl₃) δ 3.65 t, J=6.6 Hz, 0.09H), 1.62-1.58 (H₁₁, m, 0.09H), 1.40-1.00 (H_{2-5, 7-10}, m, 14H), 0.90-0.70 (H₆, s, 6H).

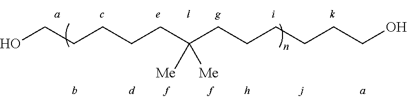

¹³C NMR (125 MHz, CDCl₃) δ 63.12 (Cₐ), 42.06, 42.02, 32.90 (Cₖ), 32.60 (Cₗ), 30.76, 30.75, 30.71, 29.83, 29.81, 27.31 (C_{f}), 24.06, 24.03.

IR (neat): 2924, 2852, 1468, 1384, 1363, 722.

### Example 3—Synthesis of Telechelic LLDPE HP(COE-s-Me₂COE)-OH 9

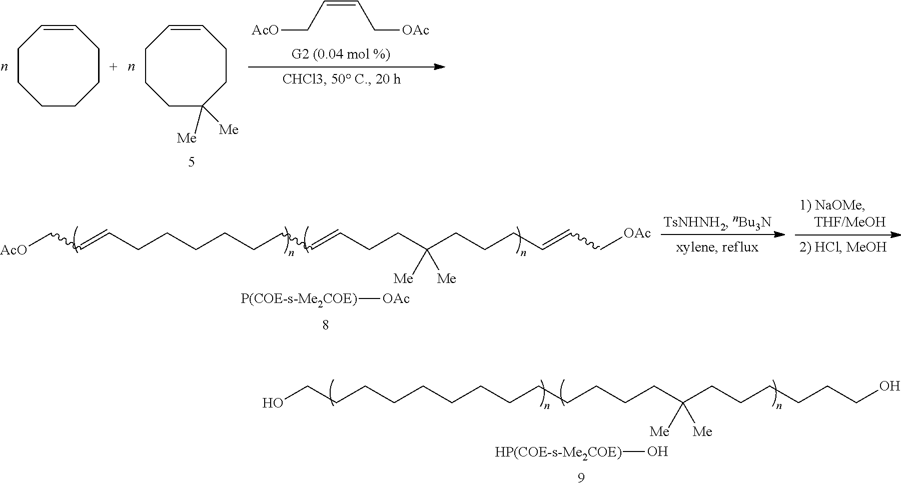

Step 1: ROMP

Following the ROMP procedure described in Example 2, 1,4-diacetoxy-cis-2-butene (47.5 μL, 0.3 mmol), cis-cyclooctene (0.66 g, 6 mmol), Me₂COE 5 (0.83 g, 6 mmol), G2 (4.1 mg, 4.8 μmol), and anhydrous CHCl₃ (4 mL) were mixed at 50° C. Upon isolation, the copolymers P(COE-s-Me₂COE)-OAc was obtained as a viscous, clear, light yellowish liquid (1.42 g, 95%).

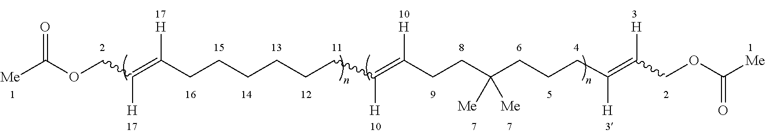

¹H NMR (500 MHz, CDCl₃) δ 5.82-5.73 (H_{3′}, m, 0.08H), 5.60-5.50 (H₃, m, 0.08H), 5.48-5.20 (H₁₀, H₁₇, m, 4H), 4.62 (H_{2-cis}, d, J=6.9 Hz, 0.02H), 4.55-4.47 (H₂₋ₜᵣₐₙₛ, m, 0.14H), 2.06 (H₁, s, 0.24H), 2.05-1.82 (H_{4,9,11,16}, m, 8H), 1.43-1.25 (H_{5,6,8,12-15}, m, 10H), 1.25-1.11 (H_{5,6,8}, m, 4H), 0.95-0.70 (H₇, two singles, 6H).

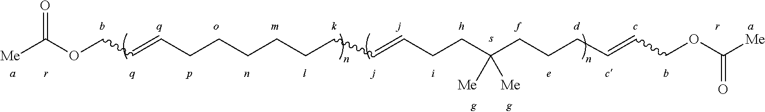

¹³C NMR (125 MHz, CDCl₃) δ 170.86 (Cᵣ), 137.44/136.72 (C_{c′}), 131.11-129.42 (C_{j,q}), 123.65/123.17 (C_{c}), 65.38/65.34 (C_{b}), 41.87, 41.48, 33.54, 33.50, 32.72, 32.61, 29.74, 29.63, 29.18, 29.05, 27.34./27.21 (C_{g}), 24.23, 24.17, 24.14, 21.05 (Cₐ).

IR (neat): 2924, 2851, 1745, 1464, 1437, 1364, 1228, 964, 723.

Step 2: Hydrogenation and Deprotection

Following the hydrogenation and deprotection procedure described in Example 2, the desired product HP(COE-s-Me₂COE)-OH was obtained as many small white solid particles (1.15 g, 91%) from 1.24 g precursor P(COE-s-Me₂COE)-OAc.

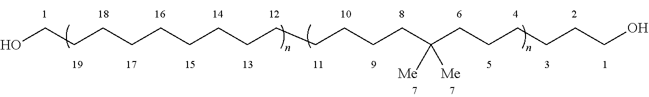

¹H NMR (500 MHz, CDCl₃) δ 3.64 (H₁, t, J=6.6 Hz, 0.16H), 1.61-1.53 (H₂, m, 0.16H), 1.40-1.05 (H₃-H₆, H₈-H₁₉, m, 30H), 0.81 (H₇, s, 6H).

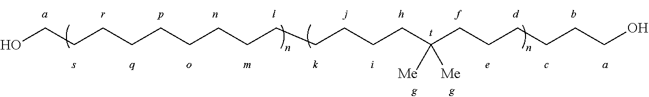

¹³C NMR (125 MHz, CDCl₃) δ 63.12 (Cₐ), 42.01, 32.83 (C_{b}), 32.58 (Cₜ), 30.75, 30.71, 29.83, 29.77, 29.73, 27.32 (C_{g}), 24.04.

IR (neat): 2919, 2849, 1467, 1364, 720.

TABLE
Characterization data of the hydroxy-telechelic polymers and unsaturated prepolymers

| | | | | | Ð | | | |
|---|---|---|---|---|---|---|---|---|
| | Mₙ (kg mol⁻¹) | | | | (M_{w}/Mₙ) | T_{g} | Tₘ | T_{d} |
| Polymer | Calc.^{c} | SEC^{d} | LS-SEC^{e} | NMR^{f} | SEC^{d} | (° C.)^{g} | (° C.)^{h} | (° C.)ⁱ |
| P(Me₂COE)-OAc^{a} | 5.0 | 6.6 | 6.6 | 5.0 | 2.00 | −56 | — | 359 |
| HP(Me₂COE)-OH^{b} | 5.0 | 8.8 | 6.9 | 5.8 | 1.61 | −45 | — | 384 |
| P(COE-s-Me₂COE)-OAc^{a} | 5.1 | 8.8 | 8.6 | 6.1 | 1.98 | −73 | −13 | 382 |
| HP(COE-s-Me₂COE)-OH^{b} | 5.1 | 9.4 | 8.2 | 6.6 | 1.97 | −37 | 31^{j} | 396 |

^{a}Targeted Mₙ = 5 kg mol⁻¹.
^{b}>99% Hydrogenation achieved.
^{c}M_{n, calc.} = (M_{w} of 5) × [5]/([CTA] + [G2]) + (M_{w} of CTA).
^{d}Determined by SEC in CHCl₃ versus polystyrene standards.
^{e}Determined by LS-SEC in THF.
^{f}Determined by NMR end-group analysis
^{g}Determined by DSC (2nd heating cycle) at 10° C. min⁻¹.
^{h}Determined by DSC (2nd heating cycle) at 2° C. min⁻¹.
ⁱ5% mass loss determined by TGA at 20° C. min⁻¹ under N₂.
^{j}This material has a broad melting transition (−15 to +77° C.).

### Example 4—Synthesis of Polyurethane 10 Based on Hydroxytelechelic HPMe₂COE-OH 7

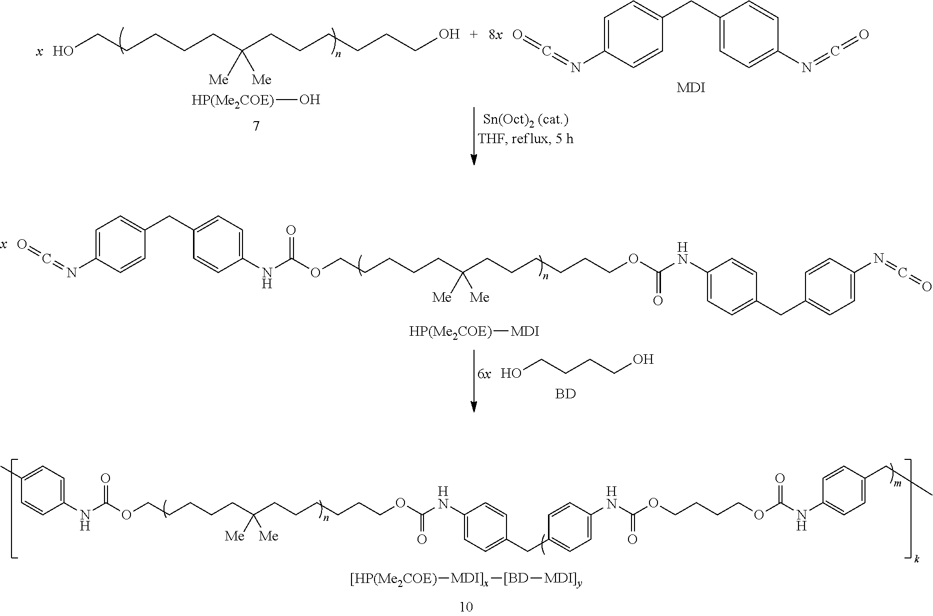

A 20 mL vial with a Teflon coated magnetic stir-bar was capped with a rubber septa. The vial was flame-dried under high vacuum then back-filled with argon; this evacuation fill cycle was repeated two more times. Methylene diphenyl diisocyanate (MDI, 200 mg, 0.8 mmol) was added to the vial and heated at 72° C. to melt. HPMe₂COE-OH (M_{n,NMR}=5.8 k) was dissolved in anhydrous THF (116 mg/mL), a solution of which (5 mL, 0.1 mmol) was added to the reaction vial and followed by the THF solution of Sn(Oct)₂ (0.1 mL, 0.002 mmol, 8.1 mg/mL). The reaction mixture was stirred at 72° C. for 4 h and then 0.5 mL solution of butandiol (BD, 54 mg, 0.6 mmol) in THF (108 mg/mL) was added to the vial. The reaction continued for 12 h at 72° C. and then terminated by adding methanol, which precipitated the desired product as off-white solids. The solids were collected by filtration and dried in vacuum oven at 45° C. for 24 h.

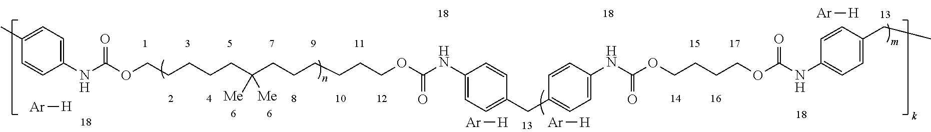

¹H NMR (500 MHz, CDCl₃/TFA-d₁=4:1) δ 7.25-7.00 (Ar—H, m, 1.62H), 4.26 (H_{1,12,14,17}, bs, 0.64H), 1.90-1.65 (H_{15,16}, two broad singles, 0.64H), 1.45-1.00 (H_{2-5,7-11} m, 14H), 0.83 (H₆, s, 6H).

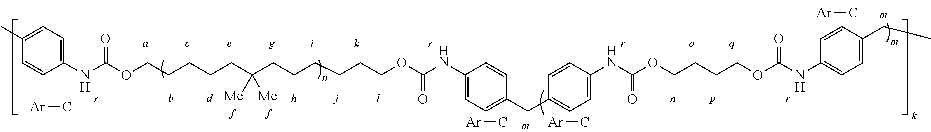

¹³C NMR (125 MHz, CDCl₃/TFA-d₁) δ 156.3-155.8 (Cᵣ), 138.2-120.5 (Ar—C), 67.4-65.5 (C_{a,l,n,q}), 42.09, 40.7-40.4 (Cm), 32.63, 30.82, 30.77, 29.90, 27.33, 25.22 (C_{o,p}), 24.12, 24.09.

IR (neat): 3325, 2925, 2852, 1702, 1529, 1468, 1310, 1228, 1078.

### Example 5—Preparation of a Polyurethane Film

A 20 ml vial with a Teflon coated magnetic stir-bar was charged with 378 mg PU 10 and 1 mL mixing solvent of CHCl₃ and TFA (90:10 in volume), and the mixture was stirred until all the solid was dissolved (around 30 min) and a very viscous solution was formed. The stir-bar was then removed and the solvent evaporated overnight at room temperature to form a film with a loose cap on the vial. The film was further dried under high vacuum at 30° C. for 2 day to give a tough, free-standing, elastic PU film.

### Example 6—Synthesis of Hydroxyl-Telechelic LLDPE HP(Me₂COE)-OH 13

The prepolymer was synthesized according to the following reaction scheme:

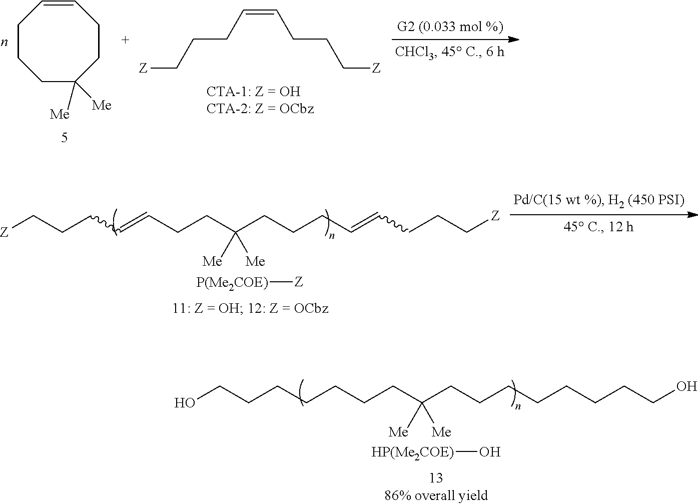

Chain transfer agents CTA-1 and CTA-2 were synthesized according to the following reaction scheme:

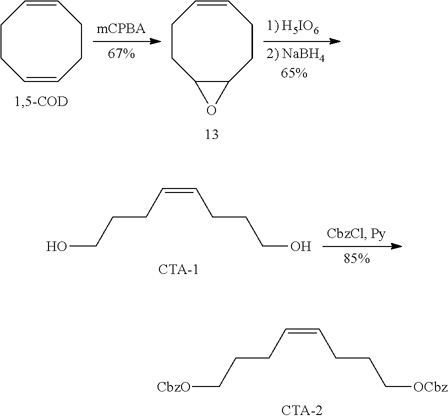

A number of embodiments of the invention have been described. Nevertheless, it will be understood that various modifications may be made without departing from the spirit and scope of the invention. Accordingly, other embodiments are within the scope of the following claims.

What is claimed is:

1. A gem-dialkyl cyclooctene monomer where each alkyl group, independently, is an acyclic alkyl group or the two alkyl groups together form a cyclic alkyl group.

2. The monomer of claim 1 wherein the monomer includes more than one pair of gem-dialkyl groups.

3. The monomer of claim 1 wherein each alkyl group, independently, is an acyclic alkyl group having between 1 and 20 carbon atoms, inclusive.

4. The monomer of claim 3 wherein each alkyl group, independently, is an acyclic alkyl group selected from the group consisting of methyl, ethyl, and propyl groups.

5. The monomer of claim 1 wherein the two alkyl groups together form a cyclic alkyl group having between 3 and 12 carbon atoms, inclusive.

6. The monomer of claim 1 wherein the monomer is a (Z)-5,5-dialkylcyclooct-1-ene monomer.

7. The monomer of claim 6 wherein each alkyl group is a methyl group.

8. A telechelic prepolymer comprising the ring opening metathesis polymerization product of a gem-dialkyl cyclooctene monomer where each alkyl group, independently, is an acyclic alkyl group or the two alkyl groups together form a cyclic alkyl group.

9. The prepolymer of claim 8 wherein the monomer includes more than one pair of gem-dialkyl groups.

10. The prepolymer of claim 8 wherein each alkyl group, independently, is an acyclic alkyl group having between 1 and 20 carbon atoms, inclusive.

11. The prepolymer of claim 8 wherein each alkyl group, independently, is an acyclic alkyl group selected from the group consisting of methyl, ethyl, and propyl groups.

12. The prepolymer of claim 8 wherein the two alkyl groups together form a cyclic alkyl group having between 3 and 12 carbon atoms, inclusive.

13. The prepolymer of claim 8 wherein the monomer is a (Z)-5,5-dialkylcyclooct-1-ene monomer.

14. The prepolymer of claim 13 wherein each alkyl group is a methyl group.

15. The prepolymer of claim 8 wherein the prepolymer comprises the ring opening polymerization product of the gem-dialkyl cyclooctene monomer and at least one cyclic olefin monomer different from the gem-dialkyl cyclooctene monomer.

16. The prepolymer of claim 15 wherein the cyclic olefin monomer has between 3 and 12 carbon atoms, inclusive.

17. The prepolymer of claim 15 wherein the cyclic olefin monomer is cis-cyclooctene.

18. The prepolymer of claim 8 wherein the prepolymer is prepared accorded to a process comprising:
(a) polymerizing the gem-dialkyl cyclooctene monomer in the presence of a ring opening metathesis polymerization catalyst and a symmetric acyclic olefin chain transfer agent having a pair of functional end groups to form an unsaturated precursor having a pair of functional end groups; and
(b) hydrogenating the precursor to form the telechelic prepolymer.

19. The prepolymer of claim 18 wherein the chain transfer agent is selected from the group consisting of

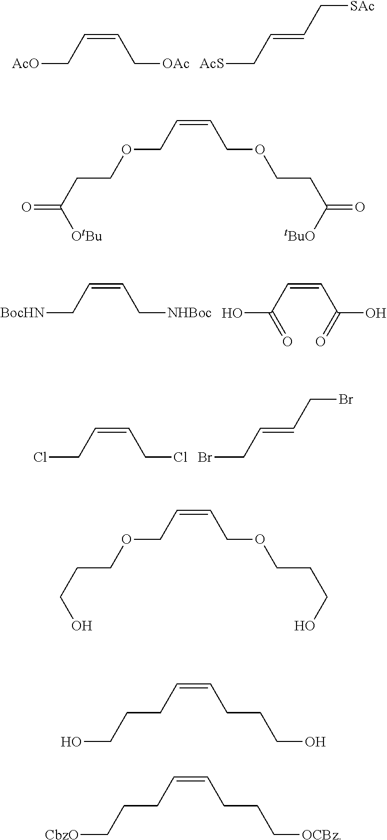

20. The prepolymer of claim 18 wherein the chain transfer agent is an unsaturated diacetoxy-functional precursor and the telechelic prepolymer is a hydroxyl-functional prepolymer.

21. A polymer comprising the reaction product of the prepolymer of claim 8 and a second polyfunctional monomer.

22. The polymer of claim 21 wherein the polymer is a polyurethane.
